(19) United States
(12) Patent Application Publication
(10) Pub. No.: US 2006/0005273 A1
(43) Pub. Date: Jan. 5, 2006

(54) Novel maize split-seed explant and methods for in vitro regeneration of maize

(75) Inventors: Sairam V. Rudrabhatla, Toledo, OH (US); Stephen L. Goldman, Toledo, OH (US); Diaa Al- Abed, Toledo, OH (US)

Correspondence Address: Catherine Martineau;MacMillian, Sobanski & Todd, LLC, One Maritime Plaza, Fourth Floor, 720 Water Street, Toledo, OH, 43604-1853, US

(73) Assignee: University of Toledo, Toledo, OH (US)

(21) Appl. No.: 11/148,754

(22) Filed: Jun. 9, 2005

Related U.S. Application Data
(60) Provisional application No. 60/578,496, filed on Jun. 10, 2004.
(60) Provisional application No. 60/643,582, filed on Jan. 14, 2005.

(51) Int. Cl. A01H 1/00; C12N 15/82; A01H 5/00

(52) U.S. Cl. 800/279; 800/289; 800/320.1

(57) ABSTRACT

The present invention provides an efficient and novel maize transformation and regeneration system based on a novel split-seed explant. Mature maize seeds are split longitudinally to form a split-seed explant. The split-seed explant can then be used in transformations to introduce a gene of interest into the maize genome to produce novel maize lines having desired characteristics. The split-seed explant can also be used to generate calli and/or multiple shoots, and rooted plantlets.

### RELATED APPLICATIONS

This application claims priority to U.S. provisional applications 60/578,496 filed Jun. 10, 2004 and 60/643,582 filed Jan. 14, 2005. Both of these provisional applications are hereby incorporated by reference in their entireties.

This invention was made, at least in part, with government support under USDA-ARS ARS Grant No. 5836071193. The U.S. government has certain rights in the invention.

### FIELD OF THE INVENTION

The present invention provides an efficient and novel maize transformation and regeneration system based on a novel split-seed explant.

### BACKGROUND OF THE INVENTION

Maize is one of the most important crops in industrialized and many developing countries. The food uses of maize, in addition to human consumption of maize kernels, include both products of dry- and wet-milling industries. Maize, including both grain and non-grain portions of the plant, is also used extensively as livestock feed, primarily for beef cattle, dairy cattle, hogs, and poultry. Therefore, there is a great demand for maize production with high quality value added traits. Hence the ability to manipulate maize in culture stems not only from the desire to elucidate the genetic control of plant development but also to exploit its commercial application.

A monocot (monocotyledon) has a single (mono) cotyledon in its seed and thus does not separate into two parts when the seed coat is removed, whereas dicots (dicotyledon) separate into two pieces when the seed coat is removed. In a monocot, the endosperm food is stored around the embryo rather than in a single seed leaf. In a dicot, the two halves are the seed leaves, or food storage areas. The initial seed leaves usually do not look like the leaves that will develop later on the growing plant.

The mature kernel of maize has three major parts: the pericarp, endosperm and embryo. See FIG. 1. (T. A. Kiesselbach 1999). The pericarp is the outer layer of the kernel, is derived from the ovary wall and is therefore genetically identical to the maternal parent. The endosperm and embryo represent the next generation. The endosperm makes up 85% of the weight of the kernel and is food source for the embryo for several days after it germinates. The embryo is located on the broad side of the kernel facing the upper end of the ear, beneath the thin layer of endosperm cells. Most of the tissue in the embryo is part of the scutellum, a spade-like structure concerned with digesting and transmitting to the geminating seedling the nutrients stored in the endosperm.

Plant regeneration from tissue culture of maize was first reported by Green and Philips (1975). In spite of this breakthrough experiment, problems related to the establishment of stable cell cultures and over coming limitations directly related to genotype dependence persisted (Tomes and Swanson, 1982, Armstrong, 1992). Recently, however Sairam et al., 2003 have shown that the totipotent cells of the shoot meristem can produce large numbers of regenerants independent of genotype, while significantly reducing the time in tissue culture.

Totipotent plant cells can undergo in vitro regeneration via two pathways: organogenesis and somatic embryogenesis. In organogenesis, totipotent cells produce a unipolar structure, namely a shoot, which is often connected to the parent tissue (Thorpe 1994). In contrast, somatic embryogenesis occurs when a bipolar structure containing a root and shoot with a closed independent vascular system are produced (Thorpe 1994).

A number of different explants have been identified in maize through which plant regeneration may occur. Specifically, maize can be regenerated in tissue culture and transformed using a variety of tissues. Explants used in previous studies include; immature embryos (Green and Philips 1975), mature embryos (Wang 1987), immature tassels (Songstad et al. 1992), coleoptilar nodes (Zhong et al. 1992a), immature inflorescences (Pareddy and Petolino 1990), glumes (Suprasanna et al. 1986), protoplasts (Prioli and Sondahl 1989; Rhodes et al. 1988a), anthers (Buter et al. 1991), microspores (Pescitelli et al 1990), leaf bases (Chang 1983), shoot tips (Zhang et al. 1992; O'Connor-Sanchez et al. 2002), shoot meristems (Sairam et al. 2003) and suspension cultures (Vasil et al. 1985). Regeneration from maize cultures was achieved through organogenesis and somatic embryogenesis (Harms et al. 1976; Potrykus et al. 1977; Rhodes et al. 1988; Vasil et al. 1984; Vasil and Vasil 1986; Prioli and Sondhal 1989; Tomes and Smith 1985; Lu et al. 1982; Novak et al. 1983; Armstrong and Green 1985).

Concomitant with the use of these regeneration protocols are severe limitations. Common problems associated with regeneration of maize from immature embryos, immature inflorescences, and embryogenic suspension culture are restrictions associated with genotype specificity, somaclonal variation, chimeras, difficulties in maintaining totipotency for extended periods of time, and low frequencies of callus induction. Moreover, all of these tissues require the constant availability of plant material and therefore these technologies have the additional disadvantage of being labor intensive. Callus-based transformation methods for corn are likewise restrictive because the regeneration from non-embryogenic (Type I) callus is very low, and the production of embryogenic (Type II) callus only occurs in the genotype A188 or its derivatives (Armstrong and Green 1985; Armstrong 1992). Finally, it is now widely accepted that the most suitable explants for transformation are those that require the least amount of time in tissue culture before and after the transformation step (Vasil 1999). This is because many studies have shown that extensive periods of tissue culture often result in somaclonal, genetic mutations, and transposon mobilization that negatively impact regenerated plants, with partial or complete sterility or loss of regeneration potential altogether.

Thus, there remains a need for a novel in vitro maize regeneration method that provides high frequency of callus induction and that doesn't require much time in tissue culture before and after transformation. The present invention fulfills this need.

### SUMMARY OF THE INVENTION

The present invention provides a novel maize explant suitable for transformation. The explant comprises a maize seed split in half longitudinally into two halves, wherein the splitting exposes the scutellum, the coleoptilar ring and shoot apical meristem, each of which are independently suitable for transformation. The maize seed is may be from an inbred cell line or a hybrid cell line.

In certain embodiments, it may be preferable to, prior to splitting the maize seed in half longitudinally, germinate the maize seed on either a either a pre-split callus priming medium comprising LS basal salts and 2,4-D or germinated on a pre-split shoot priming medium comprising MS basal salts and 2,4-D. This prior generate increases either the callus induction frequency or the shoot induction frequency.

The present invention also provides an in vitro method for transformation of maize with a gene of interest. This method involves generating a maize split-seed explant, which exposes the scutellum, the coleoptilar ring and shoot apical meristem, and transforming any one of these tissues with a gene of interest.

The present invention also provides methods of in vitro generation of at least one maize shoot from a maize split-seed explant. The at least one shoot may be either developed directly on the split-seed explant or may be developed from a callus that developed on the split-seed explant. The choice of a novel media and growing conditions (i.e. light versus darkness) dictates which fate occurs.

### BRIEF DESCRIPTION OF THE FIGURES

FIG. 1 shows a vertical section of maize split-seed explant.

FIG. 2 shows comparisons of different maize hybrid and maize inbred lines for callus induction percentages on various concentrations of 2,4-D.

FIG. 3 shows regeneration of maize plantlets from split-seed explant. FIG. 3A shows a callus induced from a split-seed explant. FIG. 3B shows callus proliferation. FIG. 3C shows embryogenic callus development. FIG. 3D shows root generation from callus. FIG. 3E shows somatic embryo development. FIG. 3F shows shoot elongation. FIGS. 3G and 3H show direct multiple shoot regenerating from a split-seed explant. FIG. 3I shows a regenerated plantlet in rooting medium. FIG. 3J shows split-seed regenerated plants in soil.

FIG. 4 shows comparisons of maize hybrid and inbred lines for multiple shoot formation on various concentrations and combinations of BAP and Kinetin.

FIG. 5A shows an isolated shoot bud originating from scutellum of a split-seed explant. FIG. 5B shows light microscopy of a cross section of a shoot bud originating from scutellum of a split-seed explant: “a” shows actively dividing cells of scutellum; “b” shows meristematic tissue originating from callus; “c” shows meristematic cells forming a shoot bud, and “d” shows a shoot bud originating from meristematic tissue.

FIG. 6 shows microscopy images of embryogenic callus and initiating shoot buds. FIG. 6A shows embryogenic callus. FIG. 6B shows actively dividing cells. FIG. 6C-6E shows a scanning electron microscope of meristematic cells grouping to form shoot buds.

FIG. 7 is a table showing the number of embryogenic calli and number of shoots regenerated per callus.

FIG. 8 is a table showing the number of shoots formed on media supplemented with BAP alone.

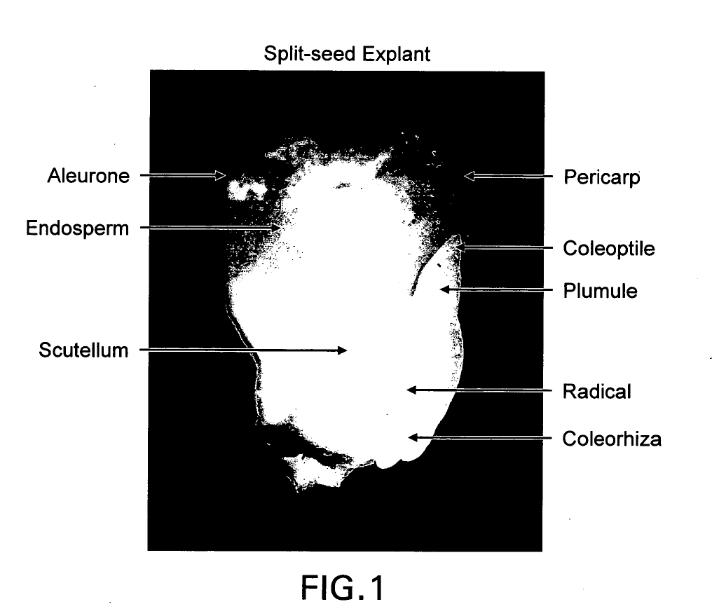

### DETAILED DESCRIPTION OF THE INVENTION

### DEFINITIONS

In order to provide a clear and consistent understanding of the specification and claims, including the scope to be given to such terms, the following definitions are provided.

“LS basal salts” is known in the art and was originally described by Linsmaier and Skoog, Physiologia Plantarum, 18:100-127 (1965). In the methods and media of the present invention, “LS basal medium” or “LS medium” or “LS basal salts” as used herein includes LS basal medium as described by Linsmaier and Skoog as well as equivalents of LS basal medium. One skilled in the art would understand that equivalents of LS basal medium include media that is substantially similar in contents and concentrations of salts, chemicals, etc., such that a tissue or plant would develop/grow in the same manner when exposed to LS basal medium. The addition of B5 vitamins is known in the art and was originally described by described by Gamborg in 1968. See O. L.; Miller, R. A.; Ojima, K., Exp. Cell Res. 50:151-158 (1968).

“MS basal salts” is known in the art and was originally described by Murashige and Skoog, Physiology Plantarum, 15:473-497 (1962). In the methods and media of the present invention, “MS basal medium” or “MS medium” or “MS basal salts” as used herein includes MS basal medium as described by Murashige and Skoog as well as equivalents of MS basal medium. One skilled in the art would understand that equivalents of MS basal medium include media that is substantially similar in contents and concentrations of salts, chemicals, etc., such that a tissue or plant would develop/grow in the same manner when exposed to MS basal medium.

MS basal salts described by Murashige and Skoog (1962) and with B₅ vitamins (“MSB₅ medium”) are as described by Gamborg, O. L.; Miller, R. A.; Ojima, K., Exp. Cell Res. 50:151-158 (1968). In the methods and media of the present invention, “MSB₅” as used herein includes MS basal medium as described by Murashige and Skoog and B₅ vitamins as described by Gamborg as well as equivalents of MSB₅. One skilled in the art would understand that equivalents of MSB₅ include media that is substantially similar in contents and concentrations of salts, chemicals, vitamins, etc. such that a tissue or plant would develop/grow in the same manner when exposed to MSB₅.

“Auxins” include, but are not limited to, naturally occurring and synthetic auxins. Naturally occurring auxin is indole acetic acid (“IAA”), which is synthesized from tryptophan. An exemplary synthetic auxin in dichlorophenoxyacetic acid (“2,4-D”). Other auxins include, but are not limited to, 4-chlorophenoxyacetic acid (“4-CPA”), 4-(2,4-dichlorophenoxy)butyric acid (“2,4-DB”), tris[2-(2,4-dichlorophenoxy)ethyl]phosphite (“2,4-DEP”), 2-(2,4-Dichlorophenoxy)propionic acid (“dicloroprop”), (RS)-2-(2,4,5-trichlorophenoxy)propionic acid (“fenoprop”), naphthaleneacetamide, α-naphthaleneacetic acid (“NAA”), 1-naphthol, naphthoxyacetic acid, potassium naphethenate, (2,4,5-trichlorophenoxy)acetic acid (“2,4,5-T”), indole-3-acetic acid, indole-3-butyric acid (“IBA”), 4-amino-3,5,6-trichloropyridine-2-carboxylic acid (“picloram”), 3,6-dichloro-o-anisic acid (“dicamba”), indole-3-proionic acid (“IPA”), phenyl acetic acid (“PAA”), benzofuran-3-acetic acid (“BFA”), and phenyl butric acid (“PBA”). A primary site of auxin production is the apical shoot meristem and the most studied function of auxin is the promotion of elongation and cell enlargement. Auxins also promote lateral and adventitious root development.

“Cytokinins” are a group of phenylurea derivatives of adenine. Cytokinins promote cytokinesis (division of the cytoplasm to a cell following the division of the nucleus). Cytokinins also retard leaf senescence. The first naturally occurring cytokinin chemically identified was called zeatin. An exemplary synthetic cytokinin is 6-benzylamino purine (“BAP”). Examples of cytokinins include, but are not limited to, 6-γ,γ-Dimethylallylaminopuine (“2iP”), kinetin, zeatin, zeatin riboside, and BAP.

“Whisker-mediated transformation” is the facilitation of DNA insertion into plant cell aggregates and/or plant tissues by elongated needle-like microfibers or “whiskers” and expression of said DNA in either a transient or stable manner. (See e.g. U.S. Pat. Nos. 5,302,523 and 5,464,765, which are herein incorporated by reference).

“Gene of interest” or may be homologous DNA, heterologous DNA, foreign DNA, genomic DNA or cDNA.

The present invention provides an in vitro method for transformation of maize with a gene of interest and also provides an in vitro method for regeneration of maize.

In both transformation and regeneration, it is an essential prerequisite to start with a tissue culture explant that exposes a greatest number of competent cells in order to achieve the maximum number of regenerants. Until recently, immature embryos have been the only reliable explant for maize regeneration, especially when coupled to transformation (Lu et al. 1982; Vasil et al. 1984). Beside the inherent difficulty of maintaining a continuous supply year round, the selection of the immature embryos at the right stage to insure predictable regeneration response is complicated.

In contrast, mature seeds can be easily stored and as such are available throughout the year to initiate tissue culture. However, mature seeds have been considered more recalcitrant to tissue culture manipulations than immature embryos based on the limited number of reports that have shown a low frequency and genotype dependent regeneration for maize mature seeds (Wang, 1987; Huang and Wei, 2004).

Contrary to what was previously believed to be possible, the present inventions utilize a mature seed to produce a tissue culture explant that is suitable for transformation. The methods of the present invention involve splitting a maize seed longitudinally into two halves to produce a split-seed explant. Split-seed explant regenerates into stronger, healthier and fertile plants. Furthermore, split-seeds are easy to handle and are available year round in bulk quantities. Additionally, in comparison with reported regeneration protocols in maize, the number of shoots and callus regeneration frequency are significantly higher than previously reported. Specifically, the number of multiple shoots regenerated directly from split-seeds via organogenesis numbered up to 28 shoots per explant. Most significantly the time needed to produce fertile plants is reduced to four months from the time of the initial explanting with seed being harvested 42 days later.

The splitting exposes three sources of undifferentiated cells from the scutellum, coleoptilar ring and shoot apical meristem. The cells from the scutellum, the coleoptilar ring and shoot apical meristem are each independently suitable for genetic transformation with a gene of interest. These cells can be simultaneously made competent to enhance the regeneration and/or increase the ability of DNA transfer. The present invention thus also provides an in vitro method for transformation of maize with gene of interest. The maize can be an inbred cell line or a hybrid cell line.

### In Vitro Method of Transformation of Maize

One embodiment of the present invention provides an in vitro method of transformation of maize. This method comprises washing mature dry seeds with antibacterial soap and surface sterilizing the seed with 70% ethanol, followed by soaking in 0.1% mercuric chloride (HgCl₂) for 7 minutes. Before the seeds are split, it is preferable to germinate them for about 48 hours on a “pre-split a callus priming medium” (comprising LS basal salts and an auxin, such as dichlorophenoxyacetic acid, commonly referred to as “2,4-D”) or for 3-4 days on a “pre-split shoot priming medium” (comprising MS basal salts and a cytokinin, such as 6-benxylamino purine, commonly referred to as “BAP”), both of which are also embodiments of the invention and are described below. The choice of medium depends on whether multiple shoots are desired (use “pre-split shoot priming medium”) or whether calli are desired (use “pre-split callus priming medium”).

After germination in either a “pre-split callus priming medium” or a “pre-split shoot priming medium,” a maize seed is split longitudinally into two halves (roughly symmetrical) with a scalpel to expose the scutellum, the coleoptilar ring and shoot apical meristems. Exposed cells of the scutellum, the coleoptilar ring or shoot apical meristems are amenable to transformation and may be transformed with a gene of interest.

A gene of interest preferably confers a desired trait such as, but not limited to, cold resistance, drought resistances, herbicide resistance, insect resistance, fungal resistance or delayed senescence. For example, DNA encoding the gene CBF (cold binding factor) or cold resistance genes isolated from deschampia Antartica or colbanthus quitensis may be used to transform the maize to generate maize plants that are resistance to cold, as well as drought. Other genes of interest, include, but are not limited to, osmotin for fungal resistance, SGT-1 for broad spectrum bacterial and fungal resistance, and VP-2 for resistance to infectious bursal disease. Additionally, genes that encode human interest proteins may also be used in the transformation. For example, the gene GAD 65 for treating type 2 diabetes may be used to transform the plants.

Any suitable method of genetic transformation may be used to transform the exposed scutellum, the coleoptilar ring or shoot apical meristems. Suitable known methods of transformation include, but are not limited to, electroporation, particle bombardment, whisker-mediated transformation and Agrobacterium-mediated transformation.

When the method of transformation comprises Agrobacterium-mediated transformation, after the maize seeds are split, the exposed tissues (scutellum, coleoptilar ring and shoot apical meristem) to be transformed are wounded. Agrobacterium-mediated transformation is then carried out by methods known by one skilled in the art. After transformation, the transformed split-seed explants are then cultured on either a “split-seed explant to callus co-cultivation medium” or a “split-seed explant to direct shoot co-cultivation medium,” both of which are also embodiments of the present invention, and are described in more detail below.

A “split-seed explant to callus co-cultivation medium” is used when generation of calli from the split-seed explant is desired. A preferred “split-seed explant to callus co-cultivation medium” comprises a LS medium supplemented with B5 vitamins, 2,4-D at 3 mg/l, 200 uM acetosyringone, L-Cysteine at 300 mg/l. The co-cultivation medium is adjusted to pH 5.3 and autoclaved at 121° C. for 20 mins. The transformed split-seed explant is incubated on the “split-seed explant to callus co-cultivation medium” for preferably three days in the dark at 25° C.

A “split-seed explant to shoot co-cultivation medium” is used when direct generation of shoots from the split-seed explant is desired. A preferred “split-seed explant to shoot co-cultivation medium” comprises a MS medium supplemented with B5 vitamins, kinetin at 2 mg/L, BAP at 4 mg/L, 200 uM Acetosyringone, and 300 mg/L cysteine. The medium is adjusted to pH 5.3 and autoclaved at 121° C. for 20 mins. The transformed split-seed explant is incubated on a “split-seed explant to shoot co-cultivation medium” for preferably three days in the dark at 25° C. Other known co-cultivation media are acceptable and may be used in the embodiments of the present invention.

After a three to four day incubation, the transformed split-seed explants are transferred either to a “split-seed explant callus induction medium” (to induce formation of calli) or to a “split-seed explant shoot induction medium” (to induce shoot formation), both of which are embodiments of the present invention and are described below.

When the method of transformation comprises biolistics, the split-seed explant is positioned so that the desired tissues (scutellum, coleoptilar ring or shoot apical meristem) are accessible to particle bombardment. After the transformation is performed, the split-seed explant is transferred to a “split-seed callus induction medium” of the present invention to allow calli formation.

Regardless of the transformation approach, using embodiments of the present invention, plants can be regenerated from a split-seed via organogenesis, somatic embryogenesis or through direct multiple shoot induction. Employing embodiments of the present invention, a large number of shoots (ie. around 28 per split-seed explant) can be produced in a very short time, many transformations can be accomplished in a very short and manageable amount of time. Hence, using somatic embryo from split-seed based callus is very efficient for any transformation approach since undifferentiated cells are reprogrammed to differentiate into somatic embryos.

### In Vitro Method of Generating Maize Shoots from Split-Seed Explant Through Embryogenic Callus/Somatic Embryo Generation

Another embodiment of the present invention provides an in vitro method of generating maize shoots from a split-seed explant. After a maize seed is germinated on a “pre-split callus priming medium” and prepared and split as described above (including if desired transformation with a gene of interest), it is exposed to a “split-seed callus induction medium,” which is an embodiment of the present invention and is described below. Exposing a split-seed explant to a “split-seed callus induction medium” results in initiating callus formation to form primary calli in about one week when the split-seed explant is cultured in the dark at 27° C.

Primary calli are then transferred biweekly for about 2-4 weeks total time to fresh “primary calli maintenance medium,” which is also an embodiment of the present invention and is described below. After about one month, primary calli become proliferated calli. Proliferated calli are then cultured on an “embryogenic callus induction medium” (which is an embodiment of the present invention and is described below) to form embryogenic calli and somatic embryos. Proliferated calli incubated in the dark at 27° C. on an “embryogenic callus induction medium” develop in about four days into embryogenic calli having somatic embryos.

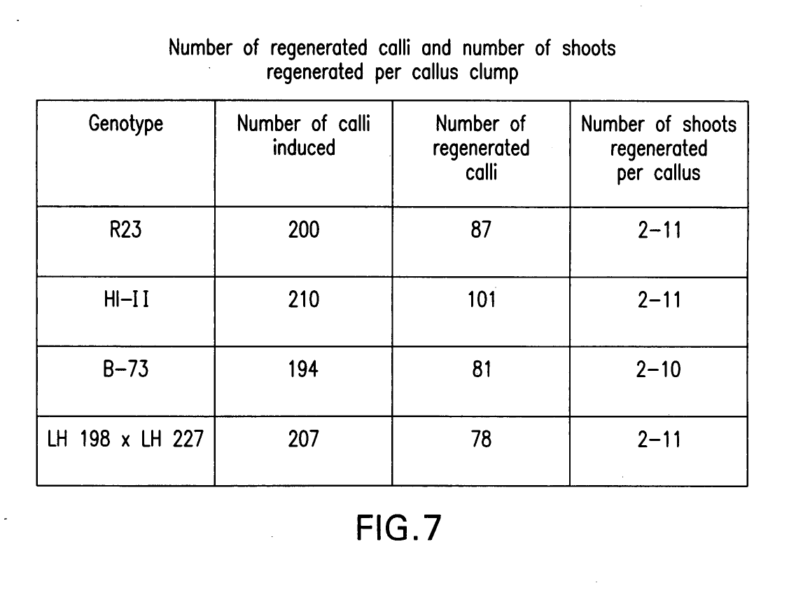

Embryogenic calli/somatic embryos are transferred to a “callus/somatic embryo shoot induction medium” (which is an embodiment of the present and is described below) and allowed to develop shoots. The cultures are maintained at 27° C. under 16-hour soft white light. Shoot regeneration frequency is determined by calculating the number of embryogenic calli producing shoots and the number of shoots per callus. See FIG. 7.

The regenerated shoots may then be transferred to a rooting medium known in the art, including, but not limited to a rooting medium comprising MS salts (Murashige and Skoog 1962) supplemented with 0.8 mg/l 1-naphthalenactic acid (“NAA”).

### In Vitro Method of Generation of a Maize Shoot from a Split-Seed Explant

Another embodiment of the invention provides a method for in vitro generation of a maize shoot, which does not involve the formation of a callus. A maize seed is germinated on a “pre-split shoot priming medium” and a split-seed explant is prepared as described above. The split-seed explant may be transformed with a gene of interest as described above and then incubated on a “split-seed explant shoot induction medium” to form a regenerated shoot. The “split-seed explant shoot induction medium,” which is an embodiment of the present invention, and is described below. The split-seed explant is incubated on a “split-seed explant shoot induction medium” under 16-h soft white light at 27° C., and allowed to develop shoots. Shoot development occurs in about three to four weeks.

### In Vitro Method of Generating a Maize Rooted Plantlet

Another embodiment of the present invention provides an in vitro method of generating a maize rooted plantlet. After a split-seed explant has developed shoots as described above (either through direct shoot induction or through calli-shoot induction), the shoot is allowed to grow for about three to four weeks. The maize shoot is then exposed to a shoot elongation medium and allowed to elongate. Shoot elongation media are known in the art and include, but are not limited to, MS basal media supplemented with B₅ vitamins. The elongated shoot is allowed to form roots and form a rooted planted by exposing the shoot to a rooting medium known in the art such as, but not limited to a rooting medium comprising MS salts and 1-naphthaleneacetic acid (“NAA”). The concentration of NAA is at about 0.5 mg/l to about 2.0 mg/l. Preferably the concentration is about 0.8 mg/l. The rooted plantlets are transferred to soil and kept in a growth chamber under 16-hour soft white light at 27° C. and 67% humidity for one week prior to transfer to the green-house.

In addition to the above embodiments of the invention, the present invention also provides various media used in the above described methods.

### Pre-Split Callus Priming Medium

The present invention provides a “pre-split callus priming medium.” Before a maize seed is split in half to generate a split-seed explant, the seed is preferably soaked for about 48 hours on a “pre-split callus priming medium” to “prime” the seed into developing callus later when a split-seed explant generated from the “primed” seed is later germinated on a “split-seed callus induction medium,” also an embodiment of the present invention. Germinating a maize seed on a “pre-split callus priming medium” before preparing a split-seed explant, increases callus induction frequency (the number of calli generated on a split-seed explant) over the callus induction frequency found on a split-seed explant generated from a seed not having been germinated in a “pre-split callus priming medium” prior to the seed split.

A “pre-split callus priming medium” comprises LS basal salts and an auxin or mixtures of auxins at a concentration from about 1.0 mg/l to about 3.5 mg/l. Preferably an auxin or mixtures thereof is at 1.5 mg/l to 3.5 mg/l and most preferably is 3.0 mg/l. In a preferred embodiment an auxin is 2,4-D and is present at 3.0 mg/l.

### Pre-Split Shoot Priming Medium

The present invention provides a “pre-split callus priming medium.” Before a maize seed is split in half to generate a split-seed explant, the seed is preferably soaked for about three to four days on a “pre-split shoot priming medium” to “prime” the seed into developing shoots later when a split-seed explant generated from the “primed” seed is later germinated on a “split-seed shoot induction medium,” also an embodiment of the present invention. Germinating the maize seed on a“pre-split shoot priming medium” before preparing the split-seed explant, increases the number of shoots generated on a split-seed explant as compared to the number of shoots generated on a split-seed explant generated from a seed not having been germinated in the“pre-split shoot priming medium” prior to the seed split.

A“pre-split shoot priming medium” comprises MS basal salts and an auxin or mixtures of auxins at a concentration of about 0.5 mg/l to about 3.0 mg/l. Preferably an auxin or mixtures thereof is at 1.0 mg/l to 2.5 mg/l and most preferably is 2.0 mg/l. In a preferred embodiment an auxin is 2,4-D and is present at 2.0 mg/l.

### Split-Seed Callus Induction Medium

One embodiment of the present invention provides a “pre-split callus induct media.” Split-seed explants exposed to callus induction medium will initiate callus formation and develop primary calli when incubated in the dark at 27° C. A callus induction medium comprises LS basal salts (See Linsmaier and Skoog 1965) and B₅ vitamins (See Gamborg et al. 1968), L-proline at a preferable concentration of 900 mg/l, glycine at a preferable concentration of 1 mg/l, casein hydrolysate at a preferable concentration of 250 mg/l, sucrose at a preferable concentration of 30 g/l and an auxin or mixtures of auxins. The auxin or mixtures thereof may be present at a concentration of 1.0 mg/l to 7.0 mg/l. Preferably the concentration of auxin is from about 1.0 mg/l to about 4.0 mg/l. More preferably the concentration of auxin is 1.0 mg/l to 3.5 mg/l. Most preferably the concentration of auxin is about 3.0 mg/l. In preferred embodiments, an auxin comprises 2,4-D and is present at about 3.0 mg/l.

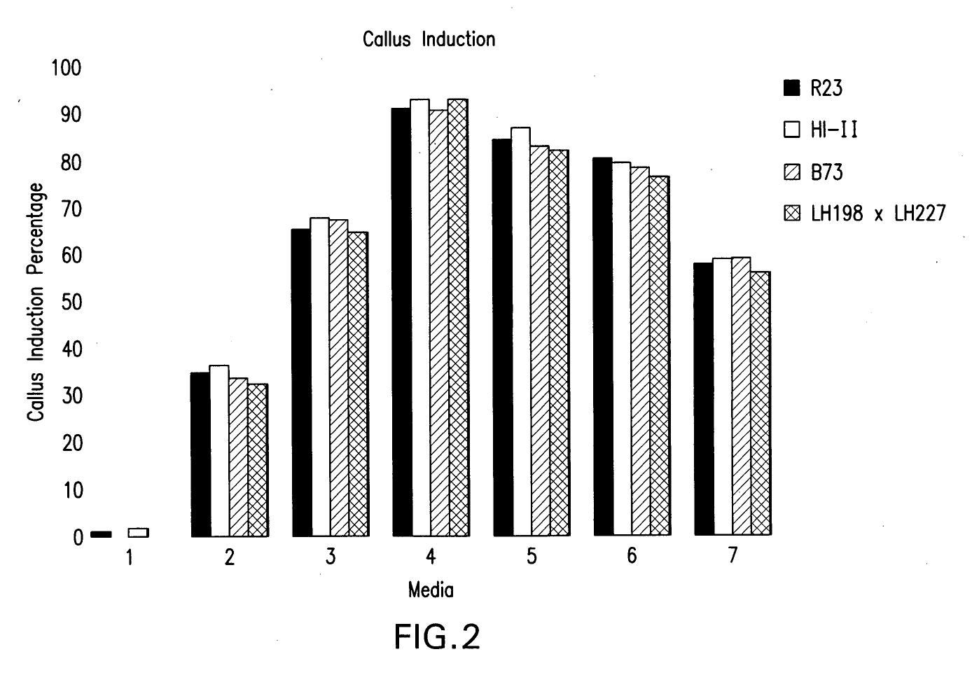

Varying concentrations of 2,4-D effect callus induction percentages. See FIG. 2. Thus, the term“about” means that the concentration need not be exactly the stated concentration but may vary, as long as the concentration provides the callus induction percentage desired. The callus induction medium may be solidified with 8.0 g/l agar. The pH is adjusted to 5.8 prior to adding the agar and the media is autoclaved at 121° C. for 20 minutes

Callus induction frequency ranges from 32% to 95.5% as a fimction of the 2,4-D concentration. See FIG. 2. After seven days of incubation on callus proliferation medium, callus induction frequency was recorded. Callus induction frequency was calculated by recording the number of split-seeds producing calli. The results recorded in FIG. 2 demonstrate that concentrations of 2,4-D from 1.0 mg/l to 7.0 mg/l induced calli. The number of explants induced callus was increased with the increment of 2,4-D concentrations up to 3.0 mg/l. A few calli were induced from B73 and R23 inbred lines, in the absence of 2,4-D. FIG. 2 also indicates that as the concentration of 2,4-D increases over 6.0 mg/l, the callus induction percentages begin to decline. With increasing 2,4-D concentrations, the appearance of the explant darkens and callus growth stops and started to be lethal at higher than 4.0 mg/l. It has been suggested that higher concentrations of 2,4-D may cause mutations that in turn kills the somatic cells (Choi et al. 2001; Vasil and Vasil 1985).

FIG. 2 also indicates that even with the same concentration of 2,4-D, there is a slight variation in callus induction percentage in different maize inbred and hybrid lines.

### Primary Calli Maintenance Medium

Another embodiment of the present invention provides a “primary calli maintenance medium.” After primary calli are formed on a split-seed explant, and after they are allowed to develop for about a week, they are incubated on a “primary calli maintenance medium” to develop into proliferated calli. A “primary calli maintenance medium” comprises LS basal salts, B₅ vitamins supplemented with an auxin, or mixtures of auxins at a concentration from about 0.5 mg/l to about 2.5 mg/l. Preferably the auxin is present at a concentration of 1.0 mg/l to 2.0 mg/l, and in preferred embodiments the auxin is 2,4-D.

### Embryogenic Callus Induction Medium

Another embodiment of the present invention provides an “embryogenic callus induction medium” comprising LS basal salts and B₅ vitamins supplemented with an auxin, or mixtures of auxins, and a cytokinin, or mixtures of cytokinins. In preferred embodiments, an auxin is 2,4-D and a cytokinin is benzylaminopurine (“BAP”). When proliferated calli are exposed to an “embryogenic callus induction medium” in the dark, they develop embryogenic calli and develop somatic embryos. Preferably an “embryogenic callus induction medium” comprises an auxin at a concentration of about 0.1 mg/l and a cytokinin at a concentration of about 0.5 mg/l. A preferred “embryogenic callus induction medium” further comprises L-proline at a preferable concentration of 900 mg/l, glycine at a preferable concentration of 1.0 mg/l, casein hydrolysate at a preferable concentration of 250 mg/l, and sucrose at a preferable concentration of 30 g/l. In a preferred embodiment, an “embryogenic callus induction medium” comprises 2,4-D at 0.1 mg/l and BAP at 0.5 mg/l.

### Callus/Somatic Embryo Shoot Induction Medium

Another embodiment of the present invention provides a “callus/somatic embryo shoot induction medium.” When an embryogenic callus/somatic embryo generated from a split-seed explant is exposed to “callus/somatic embryo shoot induction medium” under a 16-h soft white light at 27° C., the embryogenic callus/somatic embryo generates at least one shoot.

A “callus/somatic embryo shoot induction medium” comprises MS basal salts and B₅ vitamins supplemented with a cytokinin, or mixtures of cytokinins. A preferred cytokinin is BAP. The concentration of a cytokinin preferably ranges from 0.1 mg/l to 2.0 mg/l. Preferably the concentration of a cytokinin ranges from 0.5 mg/l to 2.5 mg/l and most preferably ranges from 0.75 mg/l to 1.0 mg/l. In a preferred embodiment, a cytokinin is BAP is preferably at a concentration of 1.0 mg/l.

A “callus/somatic embryo shoot induction medium” further comprises glycine at a preferable concentration of 1.0 mg/l, casein hydrolysate at a preferable concentration of 400 mg/l, and sucrose at a preferable concentration of 30 g/l. A shoot induction medium may be solidified with 8.0 g/l agar. The pH of the medium is adjusted to 5.8 prior to adding the agar and the medium is autoclaved at 121° C. for 20 minutes.

### Split-Seed Explant Shoot Induction Medium

Another embodiment of the present invention provides a “split-seed explant shoot induction medium.” When a split-seed explant is exposed to a “split-seed explant shoot induction medium” and incubated under a 16-h soft white light at 27° C., the split-seed explant generates at least one shoot. A “split-seed explant shoot induction medium” comprises MS basal salts and B₅ vitamins supplemented with a cytokinin or mixtures of cytokinins. A preferred cytokinin is BAP. The concentration of a cytokinin may range from 1.0 mg/l to 6.0 mg/l. Preferably the concentration of a cytokinin ranges from 1.0 mg/l to 5.0 mg/l and more preferably ranges from 1.5 mg/l to 4.5 mg/l. In a preferred embodiment, a cytokinin is BAP and is at a concentration of 3.0 mg/l to 4.0 mg/l. In a preferred embodiment, BAP is preferably at a concentration of 4.0 mg/l. A “split-seed explant shoot induction medium” further comprises glycine at a preferable concentration of 1.0 mg/l, casein hydrolysate at a preferable concentration of 400 mg/l, and sucrose at a preferable concentration of 30 g/l. A “split-seed explant shoot induction medium” may be solidified with 8.0 g/l agar. The pH of the medium is adjusted to 5.8 prior to adding the agar and the medium is autoclaved at 121° C. for 20 minutes.

In another embodiment, a “split-seed explant shoot induction medium” further comprises 6-furfurylaminopurine (“kinetin”). Although multiple shoots develop on a “split-seed explant shoot induction medium” comprising BAP, the addition of kinetin increases the number of shoots induced. Preferably kinetin is present at a concentration of about 0.5 mg/l to about 4.5 mg/l. Preferably the concentration of kinetin ranges from 1.5 mg/l to 3.5 mg/l and more preferably ranges from 1.75 mg/l to 2.5 mg/l. A preferred concentration of kinetin is 2.0 mg/l. In a preferred embodiment, a “split-seed explant shoot induction medium” comprises BAP at a concentration of 4.0 mg/l and kinetin at a concentration of 2.0 mg/l.

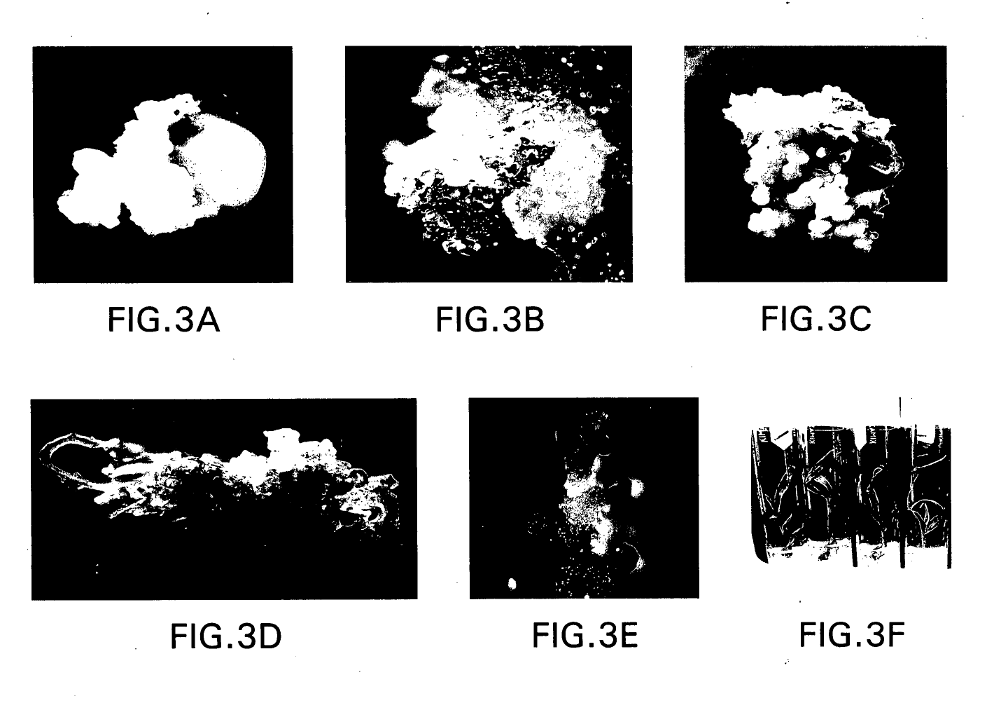

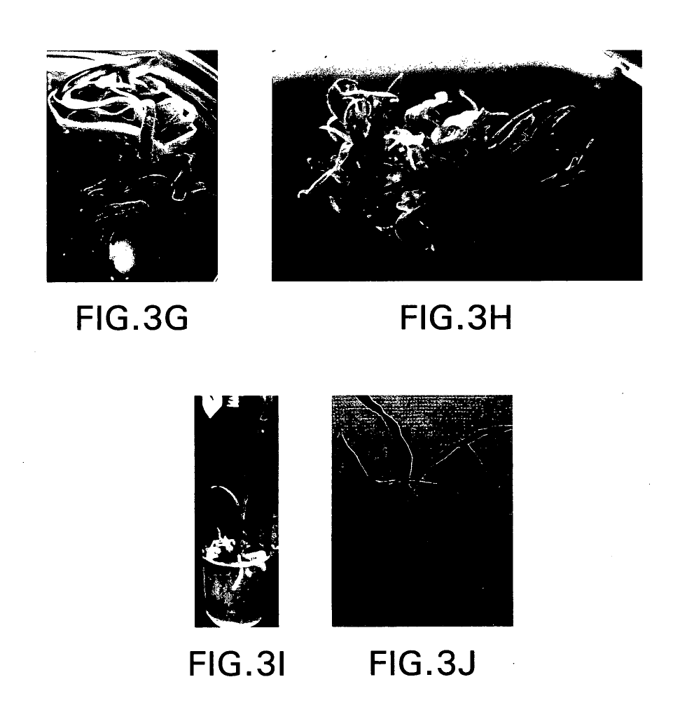

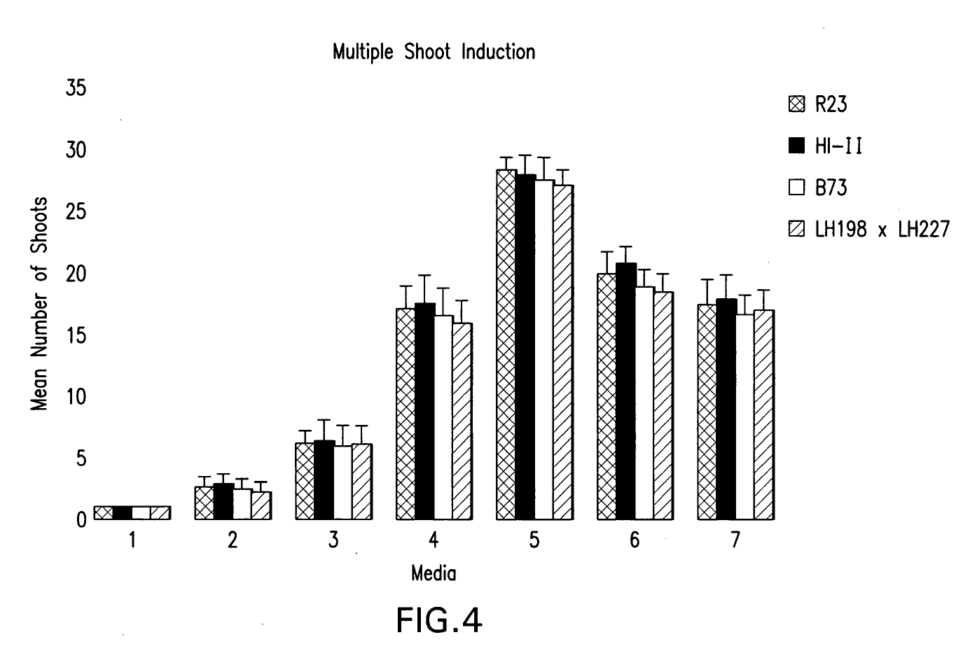

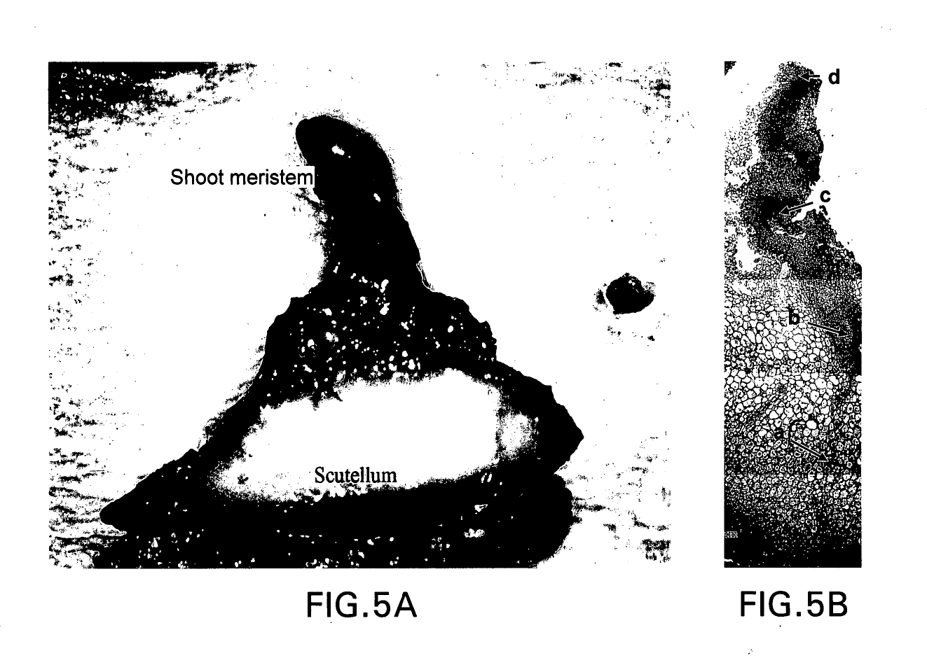

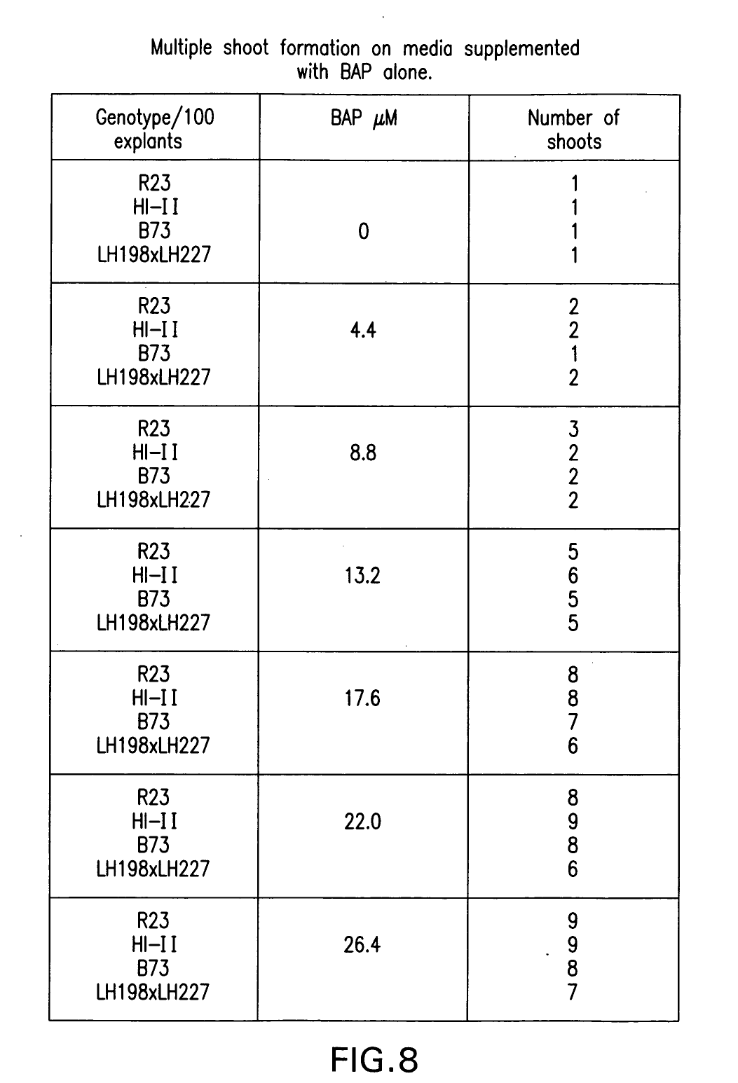

The split-seed, through organogenesis coupled with multiple shoots is genotype independent. The addition of BAP alone induces multiple shoots (FIG. 8), however the number of shoots is higher when BAP is used with the combination of kinetin. Multiple shoots are induced on media supplemented with various combinations and concentrations of BAP and kinetin (FIGS. 3G and H) and (FIG. 4). All the genotypes tested responded well to the optimal concentration of 4.0 mg/l BAP and 2.0 mg/l kinetin. The maximum number of multiple shoots per explant was nearly 28-30. Regenerated shoots may be separated and transferred to rooting media and then transferred to soil (FIGS. 3I and J).

The stage of the explants, source of light and explants pre-treatment of the seeds with a “pre-split shoot priming medium” comprising an auxin such as 2,4-D are essential factors for multiple shoot formation (data not shown). Three to four day old split-seed explants are more efficient for multiple shoot formation and provide the highest number of shoots compared to explants six or more days old. The pre-treatment of the seeds with a “pre-split shoot priming medium” comprising an auxin such as 2,4-D, has a significant effect on multiple shoot formation. When explants are not treated with “pre-split shoot priming medium,” only few explants show multiple shoots and the majority of them only germinated. Many maize reports showed that multiple shoots induction was obtained from cultures incubated in dark (Zhong et al 1992a; Lowe et al. 1995). However, in the methods of the present invention, having a light source is essential for multiple shoot induction. The highest number of shoots is obtained by incubating the explants in 16-hour soft white light at 27° C.

### EXAMPLES

### Example 1

### Preparation of Seeds and Pre-Treatment with a “Priming Medium”

Mature dry seeds of are washed with antibacterial soap and surface sterilized with 70% ethanol and soaked in 0.1% mercuric chloride (HgCl₂) for 7 minutes. For callus induction, the seeds are then rinsed several times with sterile water and soaked for 48 hours in a “pre-split callus priming medium” comprising LS (Linsmaier and Skoog 1965) liquid medium supplemented with 2,4-D at 3 mg/l.

For multiple shoot induction the seeds are soaked in sterile water for 24 hours and then germinated for three to four days on a “pre-split shoot priming medium” comprising MS (Murashige and Skoog 1962) basal salts supplemented with 2,4-D at 2 mg/l.

### Example 2

### Callus Formation and Maintenance

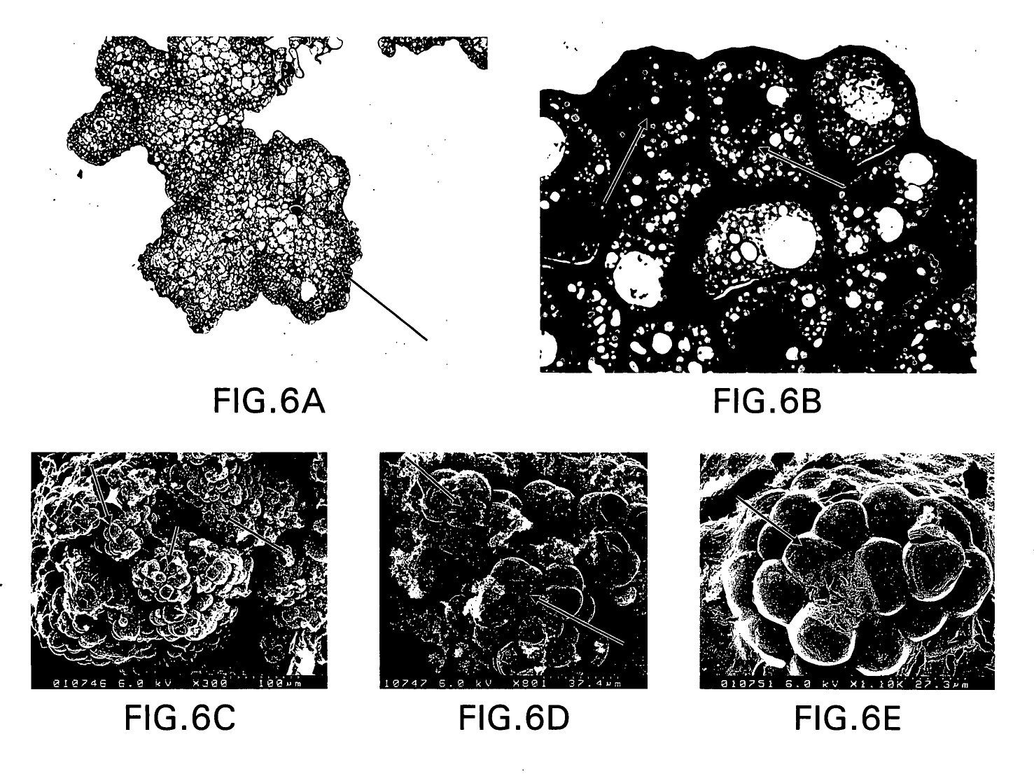

White and soft callus formed on the surface of split-seed explants is removed after one week for further growth on “primary calli maintenance medium” (FIG. 3B). Callus initiation from the split-seed is observed in four day old cultures. After one month in culture, highly proliferated calli (FIG. 3C) are transferred to an “embryogenic callus induction medium” containing 2,4-D at 0.1 mg/l and BAP at 0.5 mg/l to maintain embryogenic callus (FIG. 3D). The callus is sub-cultured every two weeks. Following the sub-culture, interestingly two types of callus are observed: embryogenic callus and organogenic callus. Organized somatic embryos are observed from the embryogenic callus (FIGS. 3D, E and F) and (FIG. 6). Direct shoot buds are also observed from the organogenic callus (FIG. 3E). Calli are further sub-cultured on a “callus/somatic embryo shoot induction medium,” which is a modified MS media supplemented with various concentrations of BAP. The number of shoots regenerated ranges from 2 to 11 shoots per each embryogenic callus. The highest number of shoots are obtained from 1.0 mg/l BAP in a maximum period of two months. Therefore, this protocol drastically reduces the time for regeneration.

### Example 3

### Multiple Shoot Formation and Plantlet Generation

Germinated mature seeds (three to four days germination) are split in half longitudinally to create split-seed explants. Split-seed explants are incubated on a “split-seed explant shoot induction medium” under 16-hour soft white light at 27° C. to allow formation of shoots. The shoots are separated from the split-seed explants after three-four weeks and incubated in a shoot elongation media containing MS basal salts and B5 vitamins. The elongated shoots are exposed to a rooting medium comprising MS basal salts supplemented with 0.8 mg/l NAA (1-naphthaleneacetic acid) to allow formation of rooted plantlets. The rooted plantlets are transferred to soil and kept in the growth chamber under 16-hour soft white light at 27° C. and 67% humidity for one week prior to transfer to the green-house.

### REFERENCES

Armstrong, C. L., and Green, C. E. (1985) Planta. 164: 207-214.
Armstrong, C. L., et al. (1992) Theor. Appl. Genet. 84: 755-762.
Bakos, A., et al. (2000) Plant Cell Rep. 19: 525-528.
Bhaskaran, S., and Smith, R. A. (1990) Crop Sci. 30: 1328-1336.
Bohorova, N. E., et al. (1995) Mayadica 40: 275-281.
Buter, B., et al. (1991) Plant Cell Rep. 10: 325-328.
Carvalho, C H. S, et al. (1997) Plant Cell Rep. 17: 73-76.
Castillo, P., et al. (2000) Plant Sci. 151: 115-119.
Chang, W. F. (1983) L. Plant Cell Rep. 2: 183-185.
Choi, H., et al. (2001) J. Plant Physiol. 158: 935-943.
Fiore, C. M., et al. (1997) Plant Cell Rep. 16: 295-298.
Gamborg, O. L., et al. (1968) Exp. Cell Res. 50: 151-158.
Gordon-Kamm, W. J., et al. (1990) Plant Cell. 2: 603-618.
Gould, J., et al. (1991) Plant. Physiol. 95: 426-434.
Green, C. E., and Philips, R. L. (1975) Crop Sci. 15: 417-421.
Harms, C. T., et al. (1976) Pflanzenzuecht. 77: 347-351.
Ishida, Y., et al. (1996) Nature Biotech. 14: 745-750.
Kiesselbach, T. A. (1999). The structure and reproduction of corn. 50^{th} anniversary edition.
Linsmaier, E., and Skoog, F. (1965) Physiol. Plant. 18: 100-127.
Lowe, K., et al. (1985). Plant Sci. 41: 125-132.
Lowe, K., et al. (1995) Bio/Technology 13: 677-681.
Lu, C., et al. (1982) Theor. Appl. Genet. 62: 109-112.
Murashige, T., and Skoog, F. (1962) Physiol. Plant. 15: 473-497.
Novak, F. J., et al. (1983) Maydica. 28: 381-390.
O'Connor-Sanchez, A., et al. (2002) Plant Cell Rep. 21: 302-312.
Pareddy, D R., and Petolino, J F. (1990) Plant Sci. 67: 211-219.
Pescitelli, S. M., et al. (1990) Plant Cell Rep. 8: 628-631.
Pasternak, T. P., et al. (1999) J. Plant Physiol. 155: 371-375.
Potrykus, I., et al. (1977) Mol. Gen. Genet. 156: 347-350.
Prioli, L. M., and Sondahl, M. R. (1989) Bio/Technology 7: 589-594.
Rhodes, C. A., et al. (1988a ) Bio/Technology 6: 56-60.
Sairam, R. V., et al. (2003) Genome 46: 323-329.
Songstad, D. D., et al. (1992) Am. J. Bot. 79: 761-764.
Suprasanna, P., et al. (1986) Theor. Appl. Genet. 72: 120-122.
Thorpe, T. A. (1994) In: Plant cell and tissue culture. Kluwer Academic Publisher, Dordrecht, pp: 17-36.
Tomes, D. T., and Smith, O. S. (1985) Theor. Appl. Genet. 70: 505-509.
Tomes, D. T. and Swanson, E. B. (1982) In: Application of plant cell and tissue culture to agriculture and industry. University of Guelph, Guelph, Ontaria, Canada, pp: 25-43.
Vasil, I. K. (1982) In: Plant tissue culture 1982 (FUJIWARA, A.), pp. 101-104. Tokyo: Maruzen.
Vasil, I. K. (1999). Molecular improvement of cereal crops. Kluwer Academic Publishers, Dordrecht.
Vasil, V., Lu, C., and Vasil, I. K. (1983) Amer J Bot. 70: 951-954.
Vasil, V., Lu, C., and Vasil, I. K. (1985) Protoplasma. 127: 1-8.
Vasil, V., and Vasil, I. K. (1986) J. Plant Physiol. 124: 399-408.
Vasil, V., et al. (1984) Am. J. Bot. 71: 158-161.
Wang, A. S. (1987). Plant Cell Rep. 6: 360-362.
Zhao Z. Y., et al. (1998) Maize Genet. Coop. Newsletter. 72: 34-37.
Zhong, H., et al. (1992a) Planta 187: 483-489.

We claim:

1. A maize explant suitable for transformation, the explant comprising a maize seed split in half longitudinally into two halves, wherein the splitting exposes the scutellum, the coleoptilar ring and shoot apical meristem, and wherein the scutellum, the coleoptilar ring and shoot apical meristem are each independently suitable for transformation.

2. The maize explant of claim 1 wherein the maize seed is from an inbred cell line or a hybrid cell line.

3. The maize explant of claim 1 wherein prior to splitting the maize seed in half longitudinally, the maize seed is germinated on either a either a pre-split callus priming medium comprising LS basal salts and 2,4-D or germinated on a pre-split shoot priming medium comprising MS basal salts and 2,4-D.

4. An in vitro method for transformation of maize with a gene of interest, the method comprising generating a maize split-seed explant comprising splitting a maize seed longitudinally into two halves to generate the split-seed explant, wherein the splitting exposes the scutellum, the coleoptilar ring and shoot apical meristem; and
wherein the scutellum, the coleoptilar ring and shoot apical meristem are each independently suitable for genetic transformation with a gene of interest; and transforming either the scutellum, the coleoptilar ring or shoot apical meristem with a gene of interest.

5. The method of claim 4, wherein the gene of interest provides a desired trait selected from the group consisting of cold resistance, drought resistances, herbicide resistance, fungal resistance, insect resistance and delayed senescence.

6. The method of claim 5, wherein the desired trait is cold resistance.

7. The method of claim 5, wherein the gene of interest encodes a CBF.

8. The method of claim 4, wherein the transformation is performed by a method selected from the group consisting of electroporation, particle bombardment, whisker-mediated transformation and Agrobacterium-mediated transformation.

9. The method of claim 4, wherein prior to splitting the maize seed, the maize seed is germinated in either a pre-split callus priming medium comprising LS basal salts and 2,4-D or germinated in a pre-split shoot priming medium comprising MS basal salts and 2,4-D.

10. A method of in vitro generation of at least one maize shoot comprising,
a) germinating a seed on a pre-split callus priming medium comprising LS basal salts and 2,4-D;
b) splitting a germinated maize seed longitudinally into two halves to generate a split-seed explant;
c) initiating primary callus formation on said split-seed explant comprising incubating the split-seed explant on a split-seed callus induction medium comprising LS basal salts, B₅ vitamins and 2,4-D to form a primary callus;
d) forming a proliferated callus comprising incubating the primary callus on a primary calli maintenance medium comprising LS basal salts, B₅ vitamins, and 2,4-D to form a proliferated callus;
e) forming an embryogenic callus comprising incubating the proliferated callus on an embryogenic callus induction medium comprising LS basal salts, B₅ vitamins, 2,4-D and BAP to form an embryogenic callus;
f) generating at least one shoot comprising incubating the embryogenic callus on a callus/somatic embryo shoot induction medium comprising MS basal salts and BAP to generate at least one shoot.

11. A method for in vitro generation of a maize shoot comprising
a) germinating a seed on a pre-split shoot priming medium comprising MS basal salts and 2,4-D;
b) splitting the germinated maize seed longitudinally into two halves to generate a split-seed explant;
c) incubating the split-seed explant on a split-seed explant shoot induction media comprising MS basal salts and BAP to generate at least one maize shoot.

12. The method of claim 11 wherein the split seed explant shoot induction media further comprises 6-furfurylaminopurine (“kinetin”).

13. An in vitro method of generating a maize rooted plantlet comprising,
a) exposing the at least one maize shoot generated in claim 10, 11 or 12 to a shoot elongation media comprising MS basal salts and B₅ vitamins, and allowing the at least one maize shoot to elongate; and
e) rooting the elongated shoot in a rooting media comprising MS basal salts and 1-naphthaleneacetic acid (“NAA”) to form a rooted plantlet.

14. A pre-split callus priming medium for germinating a maize seed before the maize seed is split in half to generate a split-seed explant, the medium comprising LS basal salts and an auxin or mixtures of auxins,
wherein the auxin or mixtures thereof is present at a concentration of 1.5 mg/l to 3.5 mg/l, and
wherein germinating said maize seed in said medium increases callus induction frequency of the split-seed explant over the callus induction frequency of a split-seed explant not having been germinated on a pre-split callus priming medium.

15. The pre-split callus priming medium of claim 14 wherein the auxin is 2,4-D and is present at 3.0 mg/l.

16. A pre-split shoot priming medium for germinating a maize seed before the maize seed is split in half to generate a split-seed explant, the medium comprising MS basal salts and an auxin or mixtures of auxins,
wherein the auxin or mixtures thereof is at a concentration of 1.0 to 3.5 mg/l, and
wherein germinating said maize seed in said medium increases shoot induction frequency of the split-seed explant over the shoot induction frequency of a split-seed explant not having been germinated on a pre-split shoot priming medium.

17. The pre-split shoot priming medium of claim 16 wherein the auxin is 2,4-D and is present at 2.0 mg/l.

18. A split seed callus induction medium comprising LS basal salts, B₅ vitamins, and 2,4-D at a concentration of 1.0 mg/l to 3.5 mg/l, wherein a split-seed explant incubated on the split seed callus induction medium in the dark generates a callus.

19. The split seed callus induction medium of claim 18 wherein 2,4-D is present at a concentration of 3.0 mg/l.

20. A primary calli maintenance medium comprising LS basal salts, B₅ vitamins, and 2,4-D at a concentration of 0.5 mg/l to 2.5 mg/l, and wherein a primary callus incubated in the dark on the primary calli maintenance medium develops into a proliferated callus.

21. An embryogenic callus induction medium comprising LS basal salts, B₅ vitamins, and 2,4-D at a concentration of 0.1 mg/l and BAP at a concentration of 0.5 mg/l, wherein when a proliferated callus developed on a maize split-seed explant is incubated on an embryogenic callus induction medium, the proliferated callus develops into an embryogenic callus.

22. A callus/somatic embryo shoot induction medium comprising MS basal salts, B₅ vitamins and BAP at a concentration of 1.0 mg/l, wherein when a somatic embryo is incubated on the callus/somatic embryo shoot induction medium, the somatic embryo develops at least one shoot.

23. A split-seed explant shoot induction medium comprising MS basal salts, B₅ vitamins, and BAP at a concentration of 2.0 mg/l to 5.0 mg/l, wherein when a maize split-seed explant is incubated on the shoot induction medium, the split-seed explant generates at least one shoot.

24. The shoot induction medium of claim 23, further comprising 6-furfurylaminopurine (“Kinetin”) at a concentration of about 1.75 mg/l to about 2.5 mg/l.

25. The shoot induction medium of claim 24, wherein the concentration of Kinetin is 2.0 mg/l and the concentration of BAP is 4.0 mg/l.
